(19) United States
(12) Patent Application Publication
(10) Pub. No.: US 2026/0102352 A1
(43) Pub. Date: Apr. 16, 2026

(54) PHARMACEUTICAL SHAPED BODIES FOR DELAYED DRUG RELEASE AND A METHOD FOR THEIR PREPARATION

(71) Applicant: MORGAN STATE UNIVERSITY, Baltimore, MD (US)

(72) Inventor: Alexandr Samokhvalov, Ellicott City, MD (US)

(21) Appl. No.: 19/358,685

(22) Filed: Oct. 15, 2025

Related U.S. Application Data
(60) Provisional application No. 63/707,414, filed on Oct. 15, 2024.

(51) Int. Cl. A61K 9/20; A61K 31/52; A61K 31/7068

(52) U.S. Cl. CPC A61K 9/2054; A61K 9/2013; A61K 9/2095; A61K 31/52; A61K 31/7068

(57) ABSTRACT

A method for preparing robust pharmaceutical shaped bodies, made thereby for delayed drug release, wherein these shaped bodies are also stable in physiological media from few days to several weeks. The method includes adding powders containing active ingredients and support material components, wherein the support material is preferably metal-organic framework, adding the additional liquid components (binders), homogenization of the obtained mixture with the use of high-speed mechanical mixing, pressing to the pharmaceutical shaped bodies, and drying these shaped bodies. The obtained pharmaceutical shaped bodies can be used for long-term (from few days to several weeks) delayed release of medicinal drugs.

### GOVERNMENT RIGHTS

This invention was made with government support under Award Numbers SC3GM136647 and UL1GM118973 awarded by National Institute of General Medical Sciences of the National Institutes of Health. The government has certain rights in the invention.

### FIELD OF THE INVENTION

This invention relates to pharmaceutical shaped bodies for drug eluting implants.

### BACKGROUND OF THE INVENTION

As is known to those skilled in the art, it is often desirable in the treatment of a number of diseases to provide active pharmaceutical components (drugs) through time delayed release for both therapeutical and prophylactical purposes.

The standard chemotherapy for many diseases is conducted via systemic drug administration, which is usually intravenous or oral. The systemic drug administration operates by the principle that drug molecules should be quickly delivered to the target tissue or organ. Systemic drug administration is usually periodic, namely, the drug needs to be administered repeatedly.

However, a major problem of systemic drug administration is the “burst” effect-a quick increase of drug concentration in the blood and tissues after administration. Namely, the concentration of the drug sharply increases at first for a short period of time (minutes to few hours) to achieve and/or exceed the concentrations which are pharmaceutically active, and then it quickly decreases to the low concentrations which are pharmaceutically inactive. Hence, the active time window of action of the drug is limited by the time period of the “burst”, namely for the short period of time immediately after drug administration. Additionally, the side effects of drugs due to the “burst” effect are particularly acute in long-term treatments, such as post-surgical maintenance of cancer patients, which is needed to suppress postoperative tumor growth.

An alternative approach is using time-delayed drug release which minimizes adverse effects of the “burst”. It can be accomplished by various forms of drug-eluting implants that have certain chemical compositions. Drug release from the implants is not only delayed, but it is also local by its nature, hence it delivers the drug directly to the affected tissue.

The drug-eluting implant is often in the form of a shaped body, such as a pellet or a bead. Such shaped bodies are made of the compositions which contain the given drug (active component) and a suitable support material, and herein these compositions are denoted “support material/drug”. The shaped body inside the drug-eluting implant provides, at its insertion site, the constant or the slowly changing concentration of the released drug. Because the drug is released in the time-delayed fashion and locally, a higher fraction of the drug is utilized, and the undesirable “burst” effect is minimized. An additional advantage of drug-eluting implants, versus systemically and periodically administered drugs, is that a drug-eluting implant is only inserted once.

At the initial stage of drug development cycle, delayed drug release is commonly tested in-vitro in physiological buffer solution, usually phosphate buffered saline (PBS). The in-vitro performance of the pharmaceutical shaped bodies in delayed drug release is commonly assessed via drug release curves: concentration or the amount of released drug versus time.

Shaped bodies for delayed drug release and drug-eluting implants, containing these shaped bodies, are advantageous in treatment of many diseases; one of them is cancer. According to the World Health Organization, “cancer is a leading cause of death worldwide, accounting for nearly 10 million deaths in 2020, or nearly one in six deaths. The most common cancers are breast, lung, colon and rectum and prostate cancers.” The advantages of delayed drug release can be illustrated by the treatment of cancer; the use of a drug-eluting implant promises benefits when: a) the local recurrence does not warrant universal treatment by a highly morbid systemic therapy, b) the local disease is inoperable or untreatable by traditional means, or c) local control offers a less invasive surgical procedure or palliative relief.

Delayed drug release can proceed by slow drug elution from compositions “support material/drug” without the “burst” effect. One example is a composition of the FDA-approved drug-eluting Gliadel wafer designed to treat brain cancer (glioma). Gliadel wafer is essentially a pellet of about 200 milligrams in weight, which contains a small amount (few milligrams) of active component carmustine (“drug”) and the rest is inactive component (“support material”) organic polymer polifeprosan. Gliadel wafer is inserted through the mount on a skull to the location of the brain tumor, where the carmustine is slowly released from the wafer to the surrounding cancer-affected tissue. Carmustine is released in the timescale of weeks, thus providing time delayed (sustained) as well as local drug release. The polifeprosan as a support material of drug releasing Gliadel wafer plays the significant role in delaying release of carmustine.

However, in most cases cancer chemotherapy is conducted by systemic and periodic drug administration, and not by delayed or local drug release. The choice of compositions for delayed drug release by shaped bodies in chemotherapy is limited.

One type of cancer of high lethality and concern is blood cancer, and specifically acute myeloid leukemia (AML). AML is a quickly progressing disease with a 5-year survival rate below 25 %. The bone marrow, where most of the blood is made, is affected by the disease and the uncontrolled growth of cancer-affected blood cells takes place in bone marrow, and cancer-affected blood cells are delivered to the bloodstream. Amongst drugs for treating AML 6-thioguanine (6-TG) is an often-prescribed antimetabolite drug of purine structure, which has been approved by the US FDA for AML treatment in remission induction and remission consolidation therapy. As with many chemotherapy drugs, major limitation of 6-TG is its high toxicity to healthy tissues, in part due to the “burst” effect.

Current treatments of leukemia include both systemic and local drug strategies. In particular, the totally implanted vascular access devices (TIVADs) are used. TIVADs allow to bypass an oral drug administration route, and the drug is delivered locally to the affected part of the body (blood). Such devices are also titled “subcutaneously implantable access ports” and a variety of them has been utilized by clinicians to deliver fluids to the blood stream. The first limitation of the TIVADs and related devices is that they do not allow delayed drug release. Their second limitation is that they do not deliver the drug to the primary site affected by leukemia - bone marrow. This is where delayed and local release of the drug would be beneficial.

It would be desirable to develop pharmaceutical compositions “support material/drug”, which can be placed in or adjacent to either the bloodstream or bone marrow, and where delayed drug release would occur. Shaped bodies consisting of compositions “support material/drug” can be inserted, for example, inside TIVADs or similar devices.

Among cancers forming solid tumors, pancreatic cancer is particularly highly lethal and difficult to cure. Gemcitabine is a widely used anti-cancer antimetabolite drug of simple molecular structure, which belongs to the group of pyrimidine drugs. The US FDA has approved gemcitabine (Gemzar®) for intravenous chemotherapy of pancreatic and few other cancers. In addition, gemcitabine is being extensively studied both in research labs and in medicinal practice. However, in its standard (systemic and periodic) administration, gemcitabine shows severe acute toxicity to patients, in part, due to the “burst”effect.

Postoperative complications are common in the therapy of pancreatic cancer, for example after pancreaticoduodenectomy (PD) for adenocarcinoma. Additionally, there are several other cancers which need post-operative maintenance therapy: breast, bone cancer (sarcoma), colon and ovarian cancer. In addition, there are several non-cancer diseases, such as bone tuberculosis where post-operative recurrence occurs, and where post-operative maintenance therapy is needed. This is where delayed and local release of anti-cancer drugs from shaped bodies would be beneficial.

### SUMMARY OF THE INVENTION

For new drug-eluting implants, it is advantageous to develop new support materials. Metal-organic frameworks (MOFs) constitute a novel advanced type of highly porous nanostructured coordination polymers. MOFs are powdered solids which consist of metal cations and anions of organic linkers.

First, “MOF/Drug” compositions, the chemical bonds between “MOF” and “Drug” are often stronger than bonds between “conventional” support materials (organic or synthetic polymers) and drug molecules; stronger bonds favor slower release of Drug molecules. Second, MOFs feature very high nanoporosity which is not available in conventional support materials. When the size of drug molecule is close or less than the size of nanopore in MOF, the molecule of drug can be strongly entrapped in the nanopore; this decreases the rate of release of drug versus non-porous or low porosity support materials. Third, certain MOFs very slowly dissolve in physiological media which would also result in slow drug release from compositions “MOF/Drug”.

For multi-component mixtures such as those in pharmaceutical shaped bodies (drug, support material, binder, etc.), a simple kneading apparatus or an extruder with slow rotation of parts may not achieve the uniform mixing. The non-uniform mixing may adversely affect mechanical strength or stability of pharmaceutical shaped bodies, resulting in their faster disintegration in physiological solution. Fast disintegration of shaped bodies such as tablets is normally desirable for oral dosages in the form of shaped bodies such as pills.

However, fast disintegration of pharmaceutical shaped bodies is not desirable for implants with delayed drug release, where slow disintegration of shaped bodies is needed.

To achieve better mixing of components of the multi-component mixtures for forming pharmaceutical shaped bodies intended for implants with delayed drug release, using high-frequency mechanical mixers is desired. However, there are no patents on using high-frequency mixers for this purpose.

First, it would be desirable to prepare pharmaceutical shaped bodies using compositions that also contain non-toxic binder materials, which improve mechanical stability of shaped bodies, and increase their stability in physiological solutions (the time interval of their stability). However, the use of binder materials to improve stability of pharmaceutical shaped bodies for delayed drug release is not among the current approaches.

Second, it would be desirable to prepare pharmaceutical shaped bodies made of several components, using methods of mechanical high-frequency mixing of their components before pressing. However, as also described above, the use of efficient mechanical high-frequency mixers for better mixing components for preparation of pharmaceutical shaped bodies for delayed drug release is not among the current approaches.

Accordingly, the present invention describes pharmaceutical shaped bodies, which demonstrate delayed drug release over a prolonged time and have stability in physiological liquids over a prolonged time, the method for their preparation, and their chemical compositions.

More specifically, there is provided according to the invention a method for fabrication of pharmaceutical shaped bodies or precursor thereto, comprising
a) mixing a pharmaceutically active ingredient and a support material component,
b) adding a liquid binder,
c) homogenizing the mixtures by high-speed mechanical mixing,
d) pressing to form shaped bodies,
e) drying the shaped bodies,
   wherein the obtained pharmaceutical shaped bodies which show delayed drug release are stable in liquid physiological media for from few days to several weeks (two, three, four weeks or longer).

Homogenization of the compositions may be achieved inside the pressing die assembly using high-speed mechanical mixing by the component of the pressing die assembly such as its anvil, for example, when the anvil is operated as a drill bit of electric drill press. Pressing of the composition into pharmaceutical shaped body may be performed by using static hydraulic press or other kind of press.

The pharmaceutical shaped body may be dried in air, in inert gas such as nitrogen or argon, or under vacuum. The pharmaceutical shaped body preferably may take the shape of a cylindrical (disk) pellet. However, no restrictions apply to the geometry or shape of the shaped bodies of the invention which may take any conceivable shape (geometry) such as spheres, honeycombs, hollow bodies etc. The shaped bodies of the invention may be of any length, preferably greater than 0.05 mm in length or diameter. According to preferred embodiments, the size of the shaped body ranges from 1.5 mm to 10 mm.

The pharmaceutical shaped body preferably exhibits delayed drug release in liquid physiological media such as phosphate buffered saline. The pharmaceutical shaped body retains its shape in liquid physiological media during delayed drug release for a long time (more than one day).

The pharmaceutically active ingredients may include 6-thioguanine, its salt, or its pro-drugs, gemcitabine free base, its salt such as gemcitabine hydrochloride, gemcitabine phosphate, or its pro-drugs, and may include two or more active ingredients.

The support material is preferably a metal-organic framework and may include two or more components which may or may not be pharmaceutically active. The binder may be a sodium salt of carboxymethylcellulose CMC, or other compound taken as its aqueous solution, and may include two or more ingredients.

According to the invention, there is also provided a pharmaceutical composition prepared according to any of the methods described herein. The pharmaceutical composition preferably exhibits delayed or accelerated in-vitro drug release to a physiological fluid or into live cells in media and/or upon administration to a subject.

There is also provided according to the invention a drug releasing implant device comprising any one of the pharmaceutical compositions described herein.

It is specifically noted that every combination and sub-combination of the above-listed and below-described features and embodiments is considered to be part of the invention.

### BRIEF DESCRIPTION OF DRAWINGS

The foregoing summary, as well as the following detailed description of the preferred invention, will be better understood when read in conjunction with the appended drawings. For the purposes of illustrating the invention, there are shown in the drawings embodiments which are presently preferred. It should be understood, however, that the invention is not limited to the precise arrangements and instrumentalities shown. In the drawings:

FIG. 1 is a photo of a commercial pellet pressing die and anvil for preparation of pressed pellets. Left: bottom die insert; center: body of pressing die; right: top-pressing die with rubber O-rings.

FIG. 2 is a temporal trace of delayed release of 6-TG drug from the pellet of composite (DUT-4) (6-TG) to PBS at 37° C. for up to 2.9 days.

FIG. 3a is a photo of a drug-releasing pellet of composite (DUT-4)(6-TG) in a 1 L dissolution vessel of automatic dissolution apparatus (pellet is below the stirring paddle) at 60 min from start of dissolution.

FIG. 3b is a photo of a drug-releasing pellet of composite (DUT-4)(6-TG) in a 1 L dissolution vessel of automatic dissolution apparatus (pellet is below the stirring paddle) at 5820 min or about 4 days from start of dissolution.

FIG. 4a shows a release profile of gemcitabine from pressed pellets to PBS in the long timescale (7200 minutes) for the composite (MOF-253) (GemHCl).

FIG. 4b shows a release profile of gemcitabine from pressed pellets to PBS in the long timescale (7200 minutes) for pure GemHCl.

### DETAILED DESCRIPTION OF THE INVENTION

We have discovered a method for preparing robust pharmaceutical pressed bodies, which show delayed drug release and high stability in physiological liquids for a long time.

The compositions of pharmaceutical pressed bodies are characterized in that:
pharmaceutical shaped bodies contain compositions of medicinal drugs as active components, and support material components which aid in delayed drug release,
the support material components are preferably metal-organic frameworks, and
compositions also contain additional liquid components (“binders”), which aid in stability of shaped bodies.

The method for preparation of pharmaceutical pressed bodies is characterized in that:
a) the composite powder is used which contains active component “drug” and the support material,
b) alternatively, the active component “drug” and the support material, both in form of powders, are first mixed,
c) then, the “binder” is added, preferentially as aqueous solution,
d) preferentially, the obtained composition “drug” “support material” “binder” is placed inside a pressing die,
e) the obtained composition in pressing die is mixed, preferentially by mechanically rotating a die anvil at high speed,
f) then, the obtained mixed composition is pressed into a shaped body in a mechanical press,
g) the obtained pharmaceutical pressed body is dried, preferentially, in vacuum.

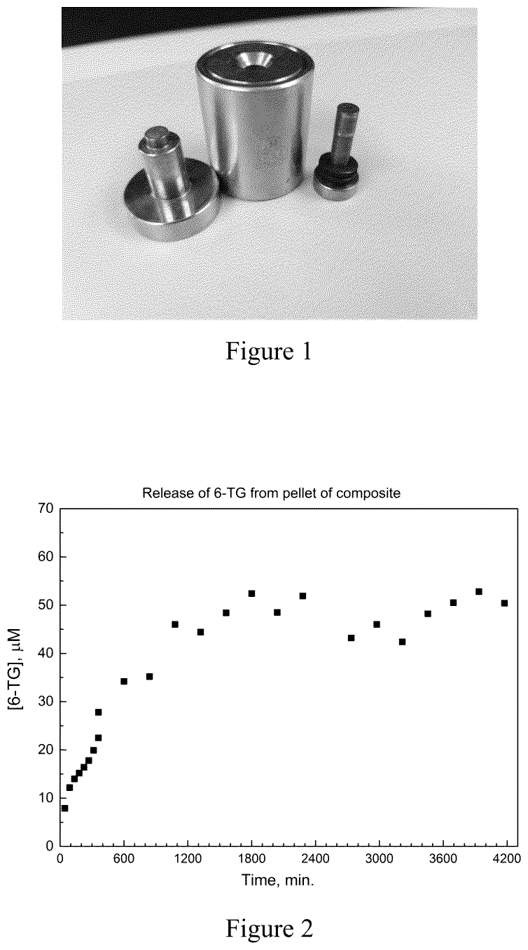

In the preferred embodiment, a commercial steel pellet pressing die assembly ¼ inch in diameter has been used (FIG. 1). The 2 % wt. aqueous solution of non-toxic binder (sodium salt of carboxymethyl cellulose) was used. First, the pellet pressing die assembly (bottom die insert and body of pressing die, but without top-pressing die (FIG. 1), was placed on the plate of semi-analytical lab balance with mass range of 0-500 g, and tared.

Second, a 20 mg solution of binder was added on top of the bottom-pressing die inside an assembly. Third, 50 mg of the powdered composite (6-TG)(DUT-4) was added. Fourth, a 20 mg solution of binder was added on top of the powder in the assembly; then, the top-pressing die with a few rubber O-rings was inserted and pressed firmly. Next, the pellet pressing die assembly with sample was fixed in a benchtop drill press. In it, the top pressing die was fixed and used as a drill bit; the content of the pellet pressing die assembly was homogenized for 5 min. by spinning the top-pressing die at a revolution rate of 300 rpm (rounds per minute). Then, a pressing assembly with a homogenized paste was placed between the plates of a benchtop hydraulic press and a pressure of 3 tons was applied. After 2 h., the pressure was released, and the pressing assembly with its content was outgassed (to evaporate water) overnight in a vacuum desiccator equipped with a manometer and a 2-stage oil-free vacuum pump (maximum flow 50 L/min, best vacuum −85 kPa). Finally, the obtained outgassed pellet was removed from the pressing assembly and stored in a closed jar, to protect it from ambient humidity.

The obtained outgassed pellet was tested for delayed release of 6-TG to phosphate buffered saline (PBS). For this, an automatic dissolution apparatus model Varian Vankel VK7000 was used, which is equipped with heater circulator model Varian Vankel VK750D, peristaltic pump, and an autosampler model VK8000. The dissolution vessel of 1 Liter in volume was filled with 750 mL PBS, maintained at 37° C. and stirred by paddle at a speed of 200 rounds per minute. After the dissolution apparatus was prepared as above, the pellet was promptly added to the dissolution vessel with PBS, and a test of delayed drug release was conducted.

In the dissolution (delayed release) test, periodically a small aliquot of 2 mL fluid from the dissolution vessel was withdrawn by an autosampler.

Then, the collected aliquots were frozen for the subsequent batch HPLC analysis. Finally, frozen aliquots of the sampled PBS with released 6-TG drug were thawed, filtered through a polytetrafluoroethylene syringe filter with pore size 0.22 μm and analyzed by an HPLC method with UV detection at wavelength 254 nm, to determine molar concentration of released 6-TG drug in solution.

The obtained pharmaceutical shaped bodies show delayed drug release and high stability in physiological buffers for a long time.

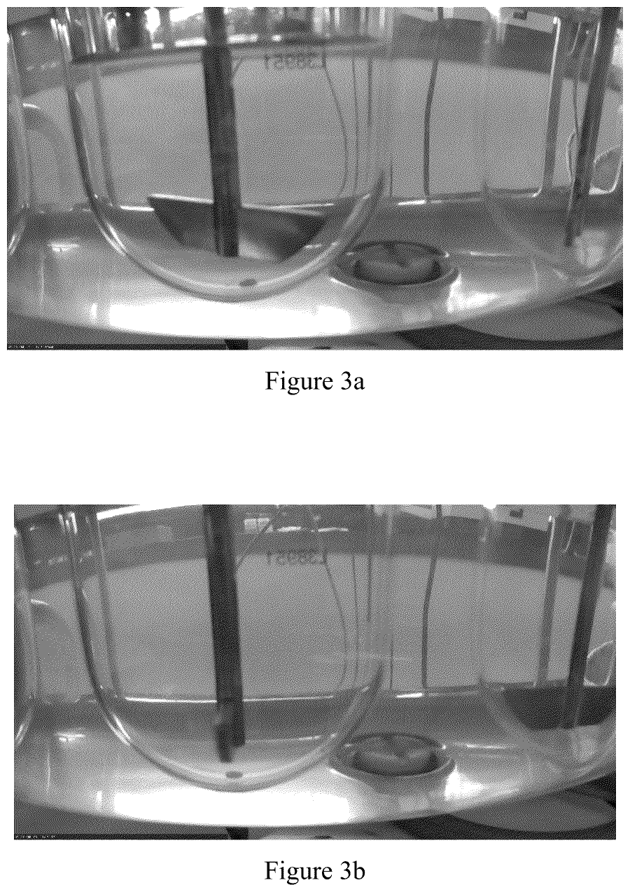

FIG. 2 shows the temporal profile of in-vitro delayed release of anti-cancer drug 6-thioguanine (6-TG) from pressed pellet made of composition (DUT-4)(6-TG). Specifically, FIG. 2 shows molar concentration of 6-TG drug released from the pellet to model physiological fluid phosphate buffered saline (PBS) at 37° C. versus time of release. Here, DUT-4 (material #4 from Dresden University of Technology) is a support material for delayed drug release, namely it is aluminum metal-organic framework (Al-MOF).

In FIG. 2 one can see that the molar concentration of drug [6-TG] increases from 1200 min until 2400 min (40 hours), and there is no “burst at the early time. This constitutes delayed drug release. Further, after [6-TG] achieves the highest value of ca. 34 μM at about 2570 min., it remains approximately constant (within ca. 5% of this value) until 4200 min (70 hours); this constitutes sustained drug release.

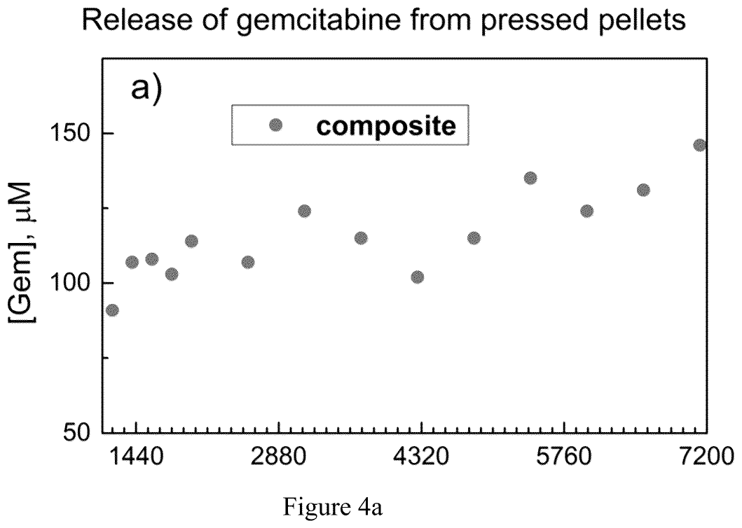

FIG. 3 shows the photographic images of this drug-releasing pellet on the bottom of cylindrical 1L dissolution vessel of automatic dissolution apparatus, below the stirring paddle. The photographic images were taken by the camera at certain time periods. In FIG. 3a (top), the pellet is shown on the bottom of the dissolution vessel in 60 min after the start of the test. In FIG. 3b (bottom) it is shown in 4 days. Thid drug-eluting pellet maintains its shape well after being stirred in PBS.

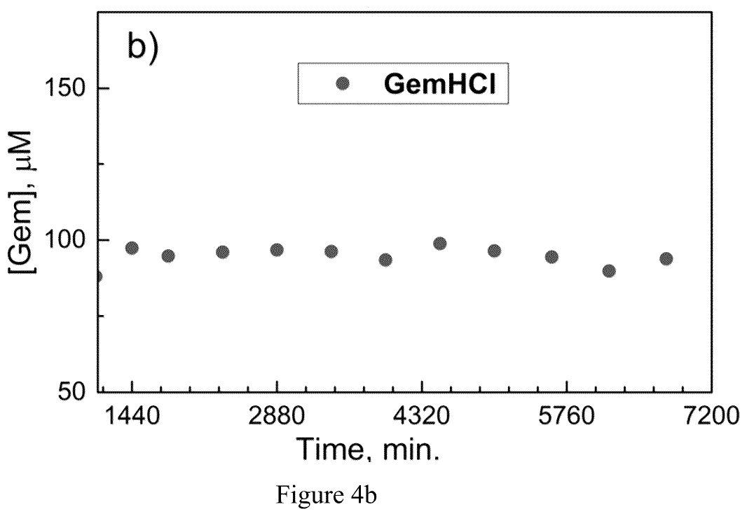

FIG. 4 shows the temporal profile of in-vitro delayed release of anti-cancer drug gemcitabine hydrochloride GemHCl from pressed pellets. In FIG. 4a, the pressed pellet made of composition (MOF-253)(GemHCl) shows a clear upward trend of concentration of gemcitabine in drug release media. MOF-253 is drug encapsulation support material. This pellet shows delayed release of gemcitabine for up to 7200 min (5 days). In contrast, in FIG. 4b, the pellet of pure GemHCl prepared by the same method yields the concentration that mostly remains constant but also shows some decrease in time.

Notwithstanding the specific embodiments, features, elements, combinations and sub-combinations disclosed herein, it is expressly considered and here disclosed that every single element, every single feature, and every combination and sub-combination thereof disclosed herein may be combined with every other element, feature, combination and sub-combination disclosed herein.

It will be appreciated by those skilled in the art that changes could be made to the embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

1. A method for fabrication of pharmaceutical shaped bodies or precursor thereto, comprising
mixing a pharmaceutically active ingredient and a support material component,
adding a liquid binder,
homogenizing the mixtures by high-speed mechanical mixing,
pressing to form shaped bodies,
drying the shaped bodies,
wherein the obtained pharmaceutical shaped bodies which show delayed drug release are stable in liquid physiological media for at least four weeks.

2. The method of claim 1 wherein homogenization of the compositions is achieved inside the pressing die assembly using high-speed mechanical mixing by the component of the pressing die assembly.

3. The method of claim 1 wherein pressing of the composition into pharmaceutical shaped body is performed by using static hydraulic press.

4. The method of claim 1, wherein pharmaceutical shaped body is dried in air, in inert gas such as nitrogen or argon, or under vacuum.

5. The method of claim 1, wherein the pharmaceutical shaped body has, preferably, the shape of a cylindrical (disk) pellet.

6. The method of claim 1, wherein the pharmaceutical shaped body shows delayed drug release to liquid physiological media.

7. The method of claim 1, wherein the pharmaceutical shaped body retains its shape in liquid physiological media during delayed drug release for at least four weeks.

8. The method of any of the preceding claims, wherein the pharmaceutically active ingredients are 6-thioguanine, its salt, or its pro-drugs.

9. The method of any of the preceding claims, wherein the pharmaceutically active ingredients are gemcitabine free base, its salt such as gemcitabine hydrochloride, gemcitabine phosphate, or its pro-drugs.

10. The method of any of the preceding claims, wherein the drug comprises two or more active ingredients.

11. The method of any one of the preceding claims wherein the support material is a metal-organic framework.

12. The method of any one of the preceding claims, wherein the support material comprises two or more components.

13. The method of any one of the preceding claims, wherein the support material consists of components that are pharmaceutically active.

14. The method of any one of the preceding claims, wherein the binder is sodium salt of carboxymethylcellulose CMC, or other compound taken as its solution.

15. The method of any one of the preceding claims, wherein the binder comprises two or more ingredients.

16. A pharmaceutical composition prepared according to the method of any one of claims 1-15.

17. The pharmaceutical composition of claim 16, wherein said pharmaceutical composition exhibits delayed or accelerated in-vitro drug release to a physiological fluid or into live cells in media.

18. The pharmaceutical composition of claim 16, wherein said pharmaceutical composition exhibits delayed or accelerated release of said drug upon administration to a subject.

19. A drug releasing implant device comprising the pharmaceutical composition of any of claims 16-18.
